(19) United States
(12) Patent Application Publication
(10) Pub. No.: US 2021/0355435 A1
(43) Pub. Date: Nov. 18, 2021

(54) CULTURE MEDIUM COMPRISING KETO ACIDS

(71) Applicant: Evonik Operations GmbH, Essen (DE)

(72) Inventors: Christian KESSLER, Aschaffenburg (DE); Andreas KARAU, Gelnhausen (DE)

(73) Assignee: Evonik Operations GmbH, Essen (DE)

(21) Appl. No.: 17/287,054

(22) PCT Filed: Nov. 7, 2019
(86) PCT No.: PCT/EP2019/080466

§ 371 Date: Apr. 20, 2021

(30) Foreign Application Priority Data
Nov. 12, 2018 (EP) 18205586.3

(51) Int. Cl. C12N 5/00

(52) U.S. Cl. CPC C12N 5/0037; C12N 2511/00; C12N 2500/90; C12N 2500/33

(57) ABSTRACT

The invention relates to a culture medium, preferably a cell culture medium, comprising at least one keto acid selected from keto leucine, keto valine, keto isoleucine and keto phenylalanine and/or a salt of these keto acids. The invention further relates to the use of a culture medium of the invention for culturing cells, preferably plant cells, animal cells or mammalian cells. Another aspect of the invention relates to a method of manufacturing a cell culture product comprising the steps of (i) providing a cell capable of producing said cell culture product; (ii) contacting said cell with a culture medium according to the invention; and (iii) obtaining said cell culture product from said culture medium or from said cell.

### FIELD OF THE INVENTION

The present invention relates to biotechnological production processes. More specifically, the present invention relates to improved culture media for use in biotechnological production processes, processes employing such improved media, and to products obtained from the processes using the improved culture media.

### BACKGROUND OF THE INVENTION

Biotechnological processes are widely used for the production of biological products. These processes typically involve the cultivation of cells in a culture medium under conditions permissible to the growth and product formation by the cultivated cells. Cells useful in biotechnological production are bacterial cells, fungal cells, yeast cells, and cells of animal or plant origin.

Animal cell culture has long been used for the production of biological products, such as therapeutic proteins, polypeptides, oligopeptides or other biological molecules, such as therapeutic polysaccharides. In such cell culture processes, animal cells, normally genetically modified to produce a desired product, are cultivated in a liquid, solid or semi solid culture medium for cell proliferation and product formation. One significant advantage of cell culture is the fact that animal cells and plant cells are able to perform post-translational modifications of the primary product, such as folding and post-translational modification of a polypeptide.

In biotechnological production processes, in particular in animal cell cultures, control and optimization of the cell culture conditions is critical for cell proliferation and product formation. One decisive factor is medium composition. The concentration and quality of the final cell culture product depends heavily on the medium composition. Animal and plant cell cultures are particularly demanding in terms of the required nutrient composition and culture conditions. Required nutrients not only include basic sources for carbon, nitrogen and energy, such as sugar and ammonia, but also more complex nutrients, such as essential amino acids, growth factors and vitamins. For this reason, supplementation of animal cell culture media with complex nutrient compositions, such as serum, has been used to provide the animal cells with a broad variety of nutrients. However, regulatory and safety concerns, as well as problems with the heterogeneity of the available sources of sera has led the industry to strive towards eliminating serum and other non-defined media from industrial cell culture processes. Cell cultures grown in serum-free media, however, often show nutritional deficiencies. For this reason, much effort has been given to identify those nutritional components of complex media, as well as their optimal concentrations, which are required for satisfactory growth and protein formation in cell cultures. A commonly used strategy to overcome nutritional deficiencies is to feed spent media components during the cultivations. The highly concentrated solutions used for replenishment are called feed media. Optimizing their composition together with the basal medium follows the same logic.

A special sub-set of application is the production of viruses either as vaccine or as a vector for gene therapy. In both cases, the cell culture process shows a distinct separation into two phases. The first phase is the manufacture of the virus, with the intention to maximize the virus yield. The second phase is the use of the vector to transfect the cells. Here, the medium ideally supports a high transfection efficiency.

The supply of cell cultures with amino acids is known to have a significant effect on growth rate and production. Glutamine is routinely used in cell culture media and feeds, since it is an important source of carbon, nitrogen and energy for the cultured cells. It was demonstrated that the amount of glutamine necessary for optimal growth of animal cell cultures is 3 to 10 times greater than the amount of other amino acids (Eagle et al., Science 130:432-37). However, glutamine as a nutrient is unstable when dissolved in water at elevated temperatures, such as heat sterilizing conditions, because pyroglutamate and ammonia are formed under heat (Roth et al. 1988, In Vitro Cellular & Developmental Biology 24(7): 696-98). For this reason, glutamate is often used in cell culture media instead of glutamine (Cell Culture Technology for Pharmaceutical and Cell-based Therapies, 52, Sadettin et al., Eds., Taylor and Francis Group, 2006).

The use of the ketone derivative of glutaric acid, alpha-ketoglutaric acid (AKG), which plays both a key role in the L-glutamine metabolism and the Krebs energy cycle, in cell culture applications has been described before (Hassel & Butler, Journal of Cell Science 96,501-508, Evonik Nutrition & Care GmbH, Scientific overview: cQrex™ AKG, February 2016). It could be demonstrated that in case of CHO and Hybridoma cell lines the maximum Viable Cell Density (VCD) could be increased by more than 30% and the Integrated Viable Cell Density (IVCD) by more than 50% by the addition of AKG to commercially available media.

While cell culture media described in the above prior art provide satisfactory performance and properties for certain cell culture processes, the prior art culture media are still not optimal. There still exists a need for further improved culture media that promote better growth and product formation in biotechnological production processes. This is especially relevant for the emerging fields of cell based virus production, either as vaccine or for therapy. Key challenges in this field are the removal of serum to improve reproductive performance and increasing virus yield in general. The latter is of special relevance for both applications. In case of a pandemic, large amounts of vaccines need to be made in a very short time. For gene therapy, minimizing the time between starting vector production and administration is a crucial parameter to ensure therapeutic success.

Therefore, it was an objective of the present invention to provide an advanced culture media, which promotes better growth and product formation for various cell lines and additionally reduces the amount of toxic ammonia.

### SUMMARY OF THE INVENTION

The above shortcomings of known culture media are addressed by the present invention. The invention is defined by the terms of the appended independent claims. Preferred embodiments of the invention are defined by the dependent claims.

The invention thus relates to a culture medium, preferably a cell culture medium, comprising at least one keto acid selected from keto leucine, keto valine, keto isoleucine and keto phenylalanine and/or a salt of these keto acids.

The invention further relates to the use of a culture medium of the invention for culturing cells, preferably plant cells, animal cells or mammalian cells.

Another aspect of the invention relates to a method of manufacturing a cell culture product comprising the steps of (i) providing a cell capable of producing said cell culture product; (ii) contacting said cell with a culture medium according to the invention; and (iii) obtaining said cell culture product from said culture medium or from said cell.

Preferred embodiments of the invention are described in further detail in the following detailed description of the invention.

### BRIEF DESCRIPTION OF THE FIGURES

FIG. 1 depicts the viral vector titer of the adeno-associated virus (AVV) type 9 (AAV9) and adeno-associated virus type 2 (AAV2) in human embryonic kidney (HEK) cells in absence and presence of a mixture of branched chain keto acid (BCKA) potassium salts according to the invention.

FIG. 2 depicts the viral vector infectivity (measured by luciferase activity) of adeno-associated virus (AVV) type 9 (AAV9) and adeno-associated virus type 2 (AAV2) in HEK cells in absence and presence of a mixture of branched chain keto acid (BCKA) potassium salts according to the invention.

FIG. 3 depicts the integrated viable cell density for the third passage of Chinese hamster ovary (CHO) cells cultivated in cell culture media containing different amounts of keto leucine instead of L-leucine.

FIG. 4 shows the relative concentration of antibody produced by the CHO cells cultivated in cell culture media containing different amounts of keto leucine instead of L-leucine.

FIG. 5 shows the maximum viable cell density of CHO cells cultivated in cell culture media containing different amounts of keto valine in addition to L-valine.

FIG. 6 shows the relative concentration of antibody produced by the CHO cells cultivated in cell culture media containing different amounts of keto valine in addition to L-valine.

### DETAILED DESCRIPTION OF THE INVENTION

Alpha-keto acids have differing functions in metabolism. The keto acid analogues of branched-chain amino acids play an important role in amino acid metabolism, especially in skeletal muscle and in the liver. One third of muscle protein consists of the branched-chain amino acids which cannot be formed by the body, but must be taken in with the diet. In the muscle, particularly in the case of physical exertion, proteins are continuously synthesized and broken down, wherein during breakdown of an amino acid the corresponding alpha-keto acid is formed by transferring the amino group to a carrier. The resultant keto acid can then be further oxidized enzymatically for energy production. The carrier is transported to the liver and there liberates ammonia which is converted into urea and excreted via the kidneys.

The use of alpha-keto acids which are derived from branched-chain amino acids for pharmaceutical purposes has long been known. For instance, alpha-keto isocaproate (keto leucine), in particular, can be used for reducing the protein breakdown in muscle and for a reduction of the formation of urea resulting from protein breakdown after muscle operations (U.S. Pat. No. 4,677,121). The use of keto leucine in malnutrition, muscular dystrophy or uremia and in other disorders which are a secondary consequence of protein breakdown in muscle is also described there. Keto leucine is administered in this case intravenously. In addition, it has been proposed to administer the alpha-keto acids of leucine, isoleucine and valine to patients who must maintain a protein-reduced diet, for example because of renal failure (U.S. Pat. No. 4,100,161). The role of alpha-keto acids within protein metabolism with respect to various medical indications is also described in Walser, M. et al., Kidney International, Vol. 38 (1990), pp. 595-604.

In the functional food sector, in contrast, the branched-chain amino acids are used directly for supporting muscle build-up, for example in athletes (Shimomura, Y. et al., American Society for Nutrition). The use of alpha-keto acids of leucine, isoleucine and valine for improving muscle performance and also for supporting muscle recovery after fatigue is described in U.S. Pat. No. 6,100,287, wherein salts of the corresponding anionic keto acids with cationic amino acids as counterion, such as, for example, arginine or lysine, are used. As a result, however, polyamines are also formed of which it is known that they can lead to apoptosis (programmed cell death). The excretion of the breakdown products of polyamines proceeds via the kidneys which are further stressed as a result.

However, no effects could be shown in cultured cells until now.

A “culture medium”, according to the invention, shall be understood as being a liquid or solid medium containing nutrients, the medium being suitable for nourishing and supporting life and/or product formation of cells in the culture. The cultured cells, according to the invention, may be bacterial cells, yeast cells, fungal cells, animal cells, such as mammalian cells or insect cells, and/or plant cells, e.g., algae. Typically, a culture medium provides essential and non-essential amino acids, vitamins, at least one energy source, lipids, and trace elements, all required by the cell for sustaining life, growth and/or product formation. The culture medium may also contain components that enhance growth and/or survival above the minimal rate, including hormones and growth factors. The culture medium has preferably a pH and a salt concentration which supports life, growth and/or product formation of the cells. A culture medium, according to the invention, preferably comprises all nutrients necessary to sustain life and proliferation of the cell culture. A culture medium can be defined or non-defined. Preferred culture media are defined culture media.

A “defined culture medium”, according to the invention, is a medium that contains no cell extracts, cell hydrolysates, or protein hydrolysates. Preferred defined media comprise no components of unknown composition. As is commonly understood by the person skilled in the art, defined media are usually free of animal-derived components. Preferably, all components of a defined medium have a known chemical structure. Preferred defined media are serum-free media. Other preferred defined media are synthetic media. Culture media other than “defined culture media” are referred to as “complex” culture media.

A “cell culture medium” shall be understood as being a culture medium suitable for sustaining life, proliferation and/or product formation of animal cells and/or plant cells. In certain embodiments, one can distinguish between a basal medium and a feed medium.

A “basal medium” shall be understood as being a solution or substance containing nutrients in which a culture of cells is initiated. A “feed medium” shall be understood as being a solution or substance with which the cells are fed after the start of the cultivation process. In certain embodiments, a feed medium contains one or more components not present in a basal medium. The feed medium can also lack one or more components present in a basal medium. Preferably, the concentration of nutrients in the feed medium exceeds the concentration in the basal medium to avoid a loss of productivity by dilution.

A “nutrient”, according to the present invention, is a chemical compound or substance which is needed by cells to live and grow. The nutrient is preferably taken up by the cell from the environment. Nutrients can be “organic nutrients” and “inorganic nutrients”. Organic nutrients include carbohydrates, fats, proteins (or their building blocks, e.g., amino acids), and vitamins. Inorganic nutrients are inorganic compounds such as, e.g., dietary minerals and trace elements. “Essential nutrients” are nutrients which the cell cannot synthesize itself, and which must thus be provided to the cell by the culture medium.

A “cell culture product”, according to the invention, shall be understood as being any useful biological compound produced by cells in cell culture. Preferred cell culture products of the invention are therapeutic proteins, diagnostic proteins, therapeutic polysaccharides, such as heparin, antibodies, e.g., monoclonal antibodies, growth factors, interleukin, peptide hormones, enzymes, and viruses including but not limited to viral vectors and vaccines. In other embodiments, including but not limited to the cultivation of stem cells for therapeutic use, the cell itself shall be understood as the cell culture product.

An “amino acid”, in the context of the present invention, shall be understood as being a molecule comprising an amino functional group (—NH2) and a carboxylic acid functional group (—COOH), along with a side-chain specific to the respective amino acid. In the context of the present invention, both alpha- and beta-amino acids are included. Preferred amino acids of the invention are alpha-amino acids, in particular the 20 “natural amino” acids as follows:

| | |
|---|---|
| Alanine | (Ala/A) |
| Arginine | (Arg/R) |
| Asparagine | (Asn/N) |
| Aspartic acid | (Asp/D) |
| Cysteine | (Cys/C) |
| Glutamic acid | (Glu/E) |
| Glutamine | (Gln/Q) |
| Glycine | (Gly/G) |
| Histidine | (His/H) |
| Isoleucine | (Ile/I) |
| Leucine | (Leu/L) |
| Lysine | (Lys/K) |
| Methionine | (Met/M) |
| Phenylalanine | (Phe/F) |
| Proline | (Pro/P) |
| Serine | (Ser/S) |
| Threonine | (Thr/T) |
| Tryptophan | (Trp/W) |
| Tyrosine | (Tyr/Y) |
| Valine | (Val/V) |

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention, is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, an oligopeptide, this shall be understood as referring to that part of the respective oligopeptide structure derived from the respective amino acid, according to the known mechanisms of biochemistry and peptide biosynthesis.

A “branched chain amino acid” (BCAA), according to the present invention, is understood as being an amino acid having an aliphatic side-chain with a branch (a central carbon atom bound to three or more carbon atoms). Among the proteinogenic amino acids, there are three BCAA: leucine, isoleucine, and valine. Non-proteinogenic BCAAs include 2-aminoisobutyric acid.

A “keto acid”, according to the present invention, is understood as being an organic compound that contains a carboxylic acid group and a ketone group.

An “alpha-keto acid” shall be understood as a keto acid having the keto group adjacent to the carboxylic acid. The keto acids according to the invention are chosen from keto analogous of amino acids, namely keto leucine, keto valine, keto isoleucine and keto phenylalanine and/or a salt of these keto acids. Salts of keto acids shall also include mixed salts of such keto acids. Such mixed salts can be produced by co-crystallization of different keto acids.

A “branched chain keto acid” (BCKA), according to the present invention, shall be understood as the keto analogous of the BCAAs, namely keto leucine, keto valine and keto isoleucine and/or salts of these BCKAs. Salts of BCKAs shall also include mixed salts of such BCKAs. Such mixed salts can be produced by co-crystallization of different BCKAs.

A “growth factor”, according to the invention, shall be understood as being any naturally occurring substance capable of stimulating cellular growth, proliferation and cellular differentiation. Preferred growth factors are in form of protein or steroid hormone. According to one embodiment of the invention, the expression “growth factor” shall be interpreted as relating to a growth factor selected from the list consisting of fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, cytokine, such as interleukins 1, 2, 6, granulocyte stimulating factor, and leukocyte inhibitory factor (LIF).

A “sterile form” of a nutrient composition, culture medium, cell culture medium, or the like, shall be understood as defining the absence of any living matter in said composition, culture medium, cell culture medium or the like.

A “solid culture medium”, in the context of the present invention, shall be understood as being any non-liquid or non-gaseous culture medium. Preferred solid culture media of the invention are gel-like culture media, such as agar-agar, carrageen or gelatine.

“Passaging of cells” (also known as subculture or splitting of cells), in the context of the present invention, is understood as meaning the transferring a small number of cells into a new culture vessel. Cells can be cultured for a longer time if they are split regularly, as it avoids the senescence associated with prolonged high cell density. Suspension cultures are easily passaged with a small amount of culture containing a few cells diluted in a larger volume of fresh media.

The present invention generally relates to a culture medium, preferably a cell culture medium, said culture medium comprising at least one keto acid selected from keto leucine, keto valine, keto isoleucine and keto phenylalanine and/or a salt of these keto acids. The keto acids are preferably selected from the branched chain keto acids keto leucine, keto valine and keto isoleucine.

In a specific embodiment, the culture medium comprises mixed salts of two or more keto acids. In this embodiment, two or more free keto acids selected from keto leucine, keto valine, keto isoleucine and keto phenylalanine are co-crystallized with one or more alkaline earth metal salts, preferably selected from calcium carbonate, calcium hydroxide, calcium acetate, calcium chloride, calcium oxide, magnesium hydroxide and magnesium acetate.

In a preferred configuration of the present invention the culture medium comprises keto leucine, keto valine and keto isoleucine in an approximate ratio of 2:1:1.

It is further preferred to use alkali metal salts or alkaline earth metal salts of the said keto acids. The preferred salts are Na⁺, K⁺, Ca²⁺ and Mg²⁺ salts of the said keto acids. Particularly preferred are the Na⁺ or K⁺ salts of the said keto acids.

In one embodiment, the culture medium further comprises at least one carbohydrate, at least one free amino acid, at least one inorganic salt, a buffering agent and/or at least one vitamin. In a particularly preferred embodiment, the culture medium comprises all of at least one carbohydrate, at least one free amino acid, at least one inorganic salt, a buffering agent and at least one vitamin.

According to another embodiment of the invention, the culture medium is in liquid form, in form of a gel, a powder, a granulate, a pellet or in form of a tablet.

In preferred embodiments, the culture medium of the invention is a defined medium, or a serum-free medium. For example, keto acids of the invention may be supplemented to the CHOMACS CD medium of Miltenyi Biotech (Bergisch Gladbach, Germany), to the PowerCHO-2 CD medium available from LONZA (Basel, Switzerland), the Acti-CHO P medium of PAA (PAA Laboratories, Pasching, Austria), the Ex-Cell CD CHO medium available from SAFC, the SFM4CHO medium and the CDM4CHO medium of ThermoFisher (Waltham, USA). The keto acids of the invention may also be supplemented to DMEM medium (Life Technologies Corp., Carlsbad, USA). The invention, however, is not limited to supplementation of the above media.

In other preferred embodiments, the culture medium is a liquid medium in 2-fold, 3-fold, 3.33-fold, 4-fold, 5-fold or 10-fold concentrated form (volume/volume), relative to the concentration of said medium in use. This allows preparation of a “ready-to-use” culture medium by simple dilution of the concentrated medium with the respective volume of sterile water. Such concentrated forms of the medium of the invention may also be used by addition of the same to a culture, e.g., in a fed-batch cultivation process.

Culture media of the present invention preferably contain all nutrients required for sustained growth and product formation. Recipes for preparing culture media, in particular cell culture media, are well known to the person skilled in the art (see, e.g., Cell Culture Technology for Pharmaceutical and Cell-Based Therapies, Öztürk and Wei-Shou Hu eds., Taylor and Francis Group 2006). Various culture media are commercially available from various sources.

The culture media of the invention preferably include a carbohydrate source. The main carbohydrate used in cell culture media is glucose, routinely supplemented at 5 to 25 nM. In addition, any hexose, such as galactose, fructose, or mannose or a combination may be used.

The culture medium typically also includes at least the essential amino acids (i.e., His, Ile, Leu, Lys, Met, Phe, Thr, Try, Val) as well as certain non-essential amino acids. A non-essential amino acid is typically included in the cell culture medium if the cell line is not capable of synthesizing the amino acid or if the cell line cannot produce sufficient quantities of the amino acid to support maximal growth. In addition, mammalian cells can also use glutamine as a major energy source. Glutamine is often included at higher concentrations than other amino acids (2-8 mM). However, as noted above, glutamine can spontaneously break down to form ammonia and certain cell lines produce ammonia faster, which is toxic.

The culture media of the invention preferably comprise salts. Salts are added to cell culture medium to maintain isotonic conditions and prevent osmotic imbalances. The osmolality of a culture medium of the invention is about 300 mOsm/kg, although many cell lines can tolerate an approximately 10 percent variation of this value or higher. The osmolality of some insect cell cultures tend to be higher than 300 mOsm/kg, and this may be 0.5 percent, 1 percent, 2 to 5 percent, 5-10 percent, 10-15 percent, 15-20 percent, 20-25 percent, 25-30 percent higher than 300 mOsm/kg. The most commonly used salts in cell culture medium include Na⁺, K⁺, Mg²⁺, Ca²⁺, Cl⁻, SO4²⁻, PO4³⁻, and HCO³⁻ (e.g., CaCl₂, KCl, NaCl, NaHCO₃, Na₂HPO₄).

Other inorganic elements may be present in the culture medium. They include Mn, Cu, Zn, Mo, Va, Se, Fe, Ca, Mg, Si, and Ni. Many of these elements are involved in enzymatic activity. They may be provided in the form of salts such as CaCl₂, Fe(NO₃)₃, MgCl₂, MgSO₄, MnCl₂, NaCl, NaHCO₃, Na2HPO4, and ions of the trace elements, such as, selenium, vanadium and zinc. These inorganic salts and trace elements may be obtained commercially, for example from Sigma (Saint Louis, Mo.).

The culture media of the invention preferably comprise vitamins. Vitamins are typically used by cells as cofactors. The vitamin requirements of each cell line vary greatly, although generally extra vitamins are needed if the cell culture medium contains little or no serum or if the cells are grown at high density. Exemplary vitamins preferably present in culture media of the invention include biotin, choline chloride, folic acid, i-inositol, nicotinamide, D-Ca⁺⁺-pantothenate, pyridoxal, riboflavin, thiamine, pyridoxine, niacinamide, A, B6, B12, C, D3, E, K, and p-aminobenzoic acid (PABA).

Culture media of the invention may also comprise serum. Serum is the supernatant of clotted blood. Serum components include attachment factors, micronutrients (e.g., trace elements), growth factors (e.g., hormones, proteases), and protective elements (e.g., antitoxins, antioxidants, antiproteases). Serum is available from a variety of animal sources including human, bovine or equine serum. When included in cell culture medium according to the invention, serum is typically added at a concentration of 5-10%(vol.). Preferred cell culture media are serum-free.

To promote cell growth in the absence of serum or in serum reduced media, one or more of the following polypeptides can be added to a cell culture medium of the invention: for example, fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, any cytokine, such as interleukins 1, 2, 6, granulocyte stimulating factor, leukocyte inhibitory factor (LIF), etc.

In other embodiments, the culture medium does not comprise polypeptides (i.e., peptides with more than 20 amino acids).

Culture media of the invention may also comprise shorter chain peptides: examples include dipeptides of glutamine, tyrosine, cysteine, cysteine, asparagine and serine.

One or more lipids can also be added to a culture medium of the invention, such as linoleic acid, linolenic acid, arachidonic acid, palmitoleic acid, oleic acid, polyenoic acid, and/or fatty acids of 12, 14, 16, 18, 20, or 24 carbon atoms, each carbon atom branched or unbranched), phospholipids, lecithin (phophatidylcholine), and cholesterol. One or more of these lipids can be included as supplements in serum-free media. Phosphatidic acid and lysophosphatidic acid stimulate the growth of certain anchorage-dependent cells, such as MDCK, mouse epithelial, and other kidney cell lines, while phosphatidylcholine, phosphatidylethanolamine, and phosphatidylinositol stimulate the growth of human fibroblasts in serum-free media. Ethanolamine and cholesterol have also been shown to promote the growth of certain cell lines. In certain embodiment, the cell culture medium does not contain a lipid.

One or more carrier proteins, such as bovine serum albumin (BSA) or transferrin, can also be added to the culture medium. Carrier proteins can help in the transport of certain nutrients or trace elements. BSA is typically used as a carrier of lipids, such as linoleic and oleic acids, which are insoluble in aqueous solution. In addition, BSA can also serve as a carrier for certain metals, such as Fe, Cu, and Ni. In protein-free formulations, non-animal derived substitutes for BSA, such as cyclodextrin, can be used as lipid carriers.

One or more attachment proteins, such as fibronectin, laminin, and pronectin, can also be added to a cell culture medium to help promote the attachment of anchorage-dependent cells to a substrate.

The culture medium can optionally include one or more buffering agents. Suitable buffering agents include, but are not limited to, N-[2-hydroxyethyl]-piperazine-N′-[2-ethanesulfonic acid] (HEPES), MOPS, MES, phosphate, bicarbonate and other buffering agents suitable for use in cell culture applications. A suitable buffering agent is one that provides buffering capacity without substantial cytotoxicity to the cells cultured. The selection of suitable buffering agents is within the ambit of ordinary skill in the art of cell culture.

Polyanionic or polycationic compounds may be added to the culture medium to prevent the cells from clumping and to promote growth of the cells in suspension.

In a preferred embodiment, the culture medium is in liquid form. The culture medium, however, can also be a solid medium, such as a gel-like medium, e.g. an agar-agar-, carrageen- or gelatine-containing medium.

Preferably, the culture medium is in sterile form.

In preferred embodiments of the invention, the keto acids are present in said culture medium in a concentration of from 0.01 to 10 g/l, or 0.1 to 5 g/l, or 0.1 to 0.5 g/l, preferably in a concentration of from 0.01 to 0.2 g/l.

The above concentrations are given as concentrations in the non-concentrated medium, i.e., the concentration as present in the actual culture. Concentrated media may include X-fold higher concentrations.

In one alternative embodiment of the present invention, the keto acids are supplemented to a culture medium containing the lacking non-keto analogous, respectively. In a specific embodiment, the culture medium comprises both keto leucine and leucine and/or both keto valine and valine and/or both keto isoleucine and isoleucine and/or both keto phenylalanine and phenylalanine. In an alternative embodiment, the amino acid is substituted by the keto analogous in the culture medium. In a specific embodiment, the culture medium comprises keto leucine instead of leucine and/or keto valine instead of valine and/or keto isoleucine instead of isoleucine and/or keto phenylalanine instead of phenylalanine.

The culture medium of the present invention can be in concentrated form. It may be, e.g., in 2-fold, 3-fold, 3.33-fold, 4-fold, 5-fold, 10-fold, 20-fold, 50-fold or 100-fold concentrated form (relative to a concentration that supports growth and product formation of the cells). Such concentrated culture media are helpful for preparing the culture medium for use by dilution of the concentrated culture medium with an aqueous solvent, such as water. Such concentrated culture media may be used in batch culture, but are also advantageously used in fed-batch or continuous cultures, in which a concentrated nutrient composition is added to an ongoing cultivation of cells, e.g., to replenish nutrients consumed by the cells during culture.

In other embodiments of the invention, the culture medium is in dry form, e.g., in form of a dry powder, or in form of granules, or in form of pellets, or in form of tablets.

The present invention also relates to the use of a culture medium of the invention for culturing cells. Another aspect of the invention relates to the use of a culture medium of the invention for producing a cell culture product.

A preferred embodiment of the invention relates to the use of a culture medium according to the invention for culturing animal cells or plant cells, most preferred mammalian cells. In specific embodiments the cells to be cultured are CHO cells, COS cells, VERO cells, BHK cells, HEK cells, HELA cells, AE-1 cells, insect cells, fibroblast cells, muscle cells, nerve cells, stem cells, skin cells, endothelial cells and hybridoma cells. Preferred cells of the invention are CHO cells and HEK cells.

Also included in the scope of the present invention is a method of culturing cells, said method comprising contacting said cells with a cell culture medium according to the invention. In one embodiment of the invention, the method of culturing cells comprises contacting the cell with a basal culture medium under conditions supporting the cultivation of the cell and supplementing the basal cell culture medium with a concentrated medium according to the present invention. In preferred embodiments, the basal culture medium is supplemented with the concentrated feed or medium on more than one day.

Another aspect relates to a method of producing a culture medium according to the invention, wherein said culture medium comprises at least one keto acid according to the invention. Methods of producing a culture medium according to the invention comprise at least one step of adding at least one keto acid of the invention to the culture medium. Likewise, an aspect of the invention relates to the use of keto acids of the invention for producing a cell culture medium.

Another aspect relates to a method of modifying a culture medium, wherein said modifying of said culture medium comprises addition of at least one keto acid of the invention to said culture medium.

Another aspect relates to a method of producing a liquid culture medium, said method comprising providing solid medium according to the invention, e.g., in form of a dry powder, or in form of granules, or in form of pellets, or in form of tablets; and dissolving said solid culture medium in an aqueous medium, such as water.

Another aspect of the invention relates to the use of keto acids according to the invention in a culture medium for culturing cells. Another aspect of the invention relates to the use of keto acids according to the invention for cell culture.

The invention also relates to methods of manufacturing a cell culture product comprising the steps of (i) providing a cell capable of producing said cell culture product; (ii) contacting said cell with a culture medium of the invention; and (iii) obtaining said cell culture product from said culture medium or from said cell. Likewise, the present invention relates to the use of keto acids according to the invention for manufacturing a cell culture product.

In preferred methods, the cell culture product is a therapeutic protein, a diagnostic protein, a polysaccharide, such as heparin, an antibody, a monoclonal antibody, a growth factor, an interleukin, virus, virus-like particle or an enzyme.

Cultivation of cells, according to the invention can be performed in batch culture, in fed-batch culture or in continuous culture.

### EXAMPLES

### Example 1

### Production of Viral Vectors for Adeno-Associated Virus (AVV) Type 9 (AAV9) and Adeno-Associated Virus Ttype 2 (AAV2) in Human Embryonic Kidney (HEK) Cells

Human embryonic kidney (HEK 293T) cells were cultivated in serum-free culture medium (FreeStyle 293 Expression Medium, Thermo Fisher Scientific) with or without addition of 1 mM of a mixture of BCKA in the potassium salt form. The mixture used comprised K⁺-salts of the branched chain keto acids keto leucine : keto isoleucine: keto valine in a ratio of 2:1:1. The cells were transfected with plasmid DNA to produce the viral vectors for the adeno-associated virus (AVV) type 9 (AAV9) and adeno-associated virus type 2 (AAV2) in presence or in absence of the mixture of branched chain keto acids. The amount of plasmids produced in the HEK 293T cells was determined using quantitative real-time polymerase chain reaction (qPCR).

For the qPCR analysis the cultivated cells were scraped from the wells and pelleted by centrifugation at 1,500 rpm for 5 min. The supernatant was removed to a remaining of 1.0 ml of medium; the cells were resuspended and frozen at −80° C. To ensure lysis of the cells, they were frozen and thawed three times. After the final thawing the cells were centrifuged at 1,000×g for 5 min and the supernatant was transferred into a new tube. To remove any free viral DNA not encapsulated and the capsid from intact viral particles, the supernatant was treated with DNAse and Proteinase K. The real-time q PCR was performed to detect the polyA region of AAV DNA.

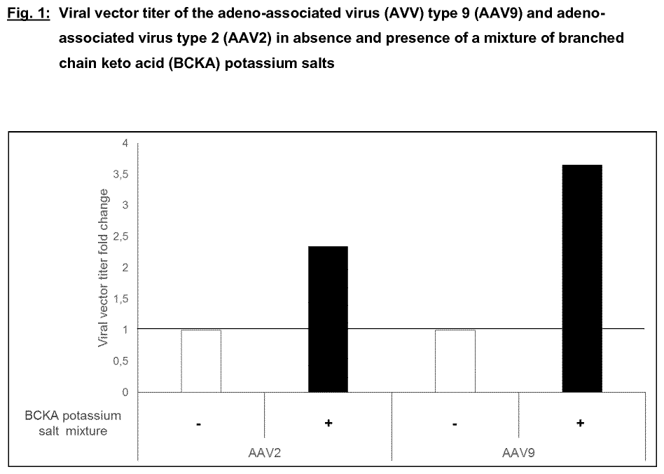

The viral vector titer of the adeno-associated virus (AVV) type 9 (AAV9) and adeno-associated virus type 2 (AAV2) in absence and presence of the mixture of branched chain keto acid (BCKA) potassium salts is shown in FIG. 1. The viral vector titer measured in serum-free culture medium without addition of BCKAs was normalized to 1 to show the change in viral vector titer in comparison to addition of BCKAs to the culture medium. Unexpectedly, addition of a mixture of BCKA potassium salts to the serum-free medium for the cultivation of cells resulted in a 2.3-fold higher viral vector titer for the AAV2 and a 3.7-fold higher viral vector titer for AAV9.

### Example 2

### Infection of Human Embryonic Kidney (HEK) Cells with Viral Vectors for Adeno-Associated Virus (AVV) Type 9 (AAV9) and Adeno-Associated Virus Type 2 (AAV2)

The virus particles produced by the transfected HEK 293T cells as described in example 1 were harvested and fresh HEK 293T cells were incubated with the virus supernatants in serum-free culture medium in absence or presence of the mixture of BCKA potassium salts also used in example 1. The infectivity of virus particles was analyzed using a luciferase assay. The luciferase gene is also incorporated in the virus vector and the luciferase activity which is detected is proportional to the amount of luciferase produced and serves as a readout for uptake of virus particles into the cells.

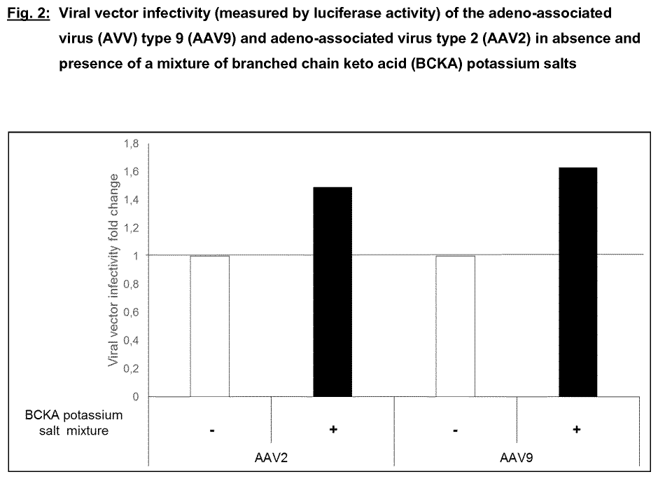

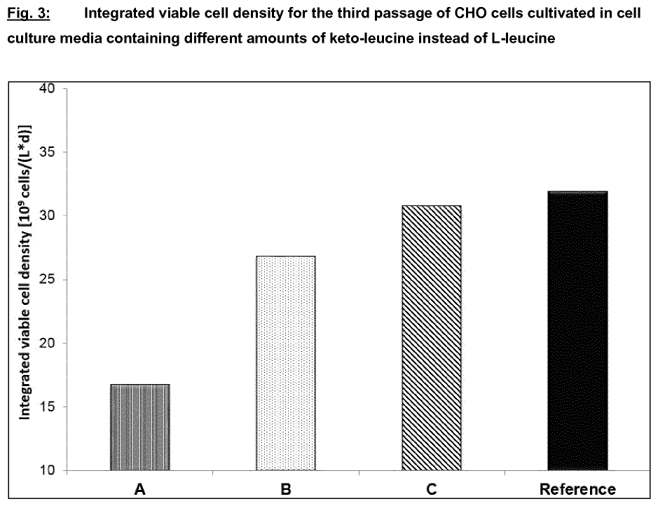

FIG. 2 shows the viral vector infectivity (measured by luciferase activity) of the adeno-associated virus (AVV) type 9 (AAV9) and adeno-associated virus type 2 (AAV2) in absence and presence of a mixture of BCKA potassium salts. The infectivity determined in serum-free culture medium without addition of BCKAs was normalized to 1 to show the change in viral vector infectivity in comparison to addition of BCKAs to the medium. Unexpectedly, addition of a mixture of BCKA potassium salts to the serum-free culture medium for the cultivation of cells resulted in a 1.5-fold increased infectivity for the AAV2 and a 1.65-fold increased infectivity for AAV9.

### Example 3

### Antibody Production in Chinese Hamster Ovary (CHO) Cells in Presence of Different Concentrations of Keto Leucine

Antibody producing chinese hamster ovary (CHO) cells (subclone DG44) were cultivated in shake flasks (125 ml or 250 ml with a culture volume of 50 ml or 100 ml respectively) in TC42/CHOMACS CD culture medium (TeutoCell AG) containing 8 mM L-glutamine. The culture medium was prepared by mixing two solutions. The first contained the conventional medium formulation (containing L-leucine and calcium chloride). In the second, the amino acid L-leucine in the culture medium was substituted by keto leucine (178 mg/l) as calcium salt in equimolar quantities. These solutions were mixed in different ratios. The growth of cells cultivated in the different mixtures was compared to cells cultivated in the conventional culture medium.

FIG. 3 shows the integrated viable cell density of CHO cells cultivated in culture media containing different amounts of keto leucine instead of L-leucine. The X-axis shows the integrated viable cell density (VCD) in 10⁶ cells per ml. A corresponds to cells cultivated in a culture medium containing 178 mg/L of keto leucine, B to a mixture containing 30% (v/v) of the keto leucine solution, C to a mixture containing 5% (v/v) of the keto leucine solution and a reference containing only L-leucine. The resulting concentrations of keto leucine in the final culture medium are shown in Table 1. The viable cell density was slightly reduced for the medium containing keto leucine instead of L-leucine. However, it could be clearly demonstrated that the amino acid L-leucine can be substituted by the keto analogue keto leucine.

TABLE 1
Keto leucine (keto-leu) concentrations in the final culture medium

| | Final keto-leu | |
|---|---|---|
| Conditions | concentration | |
| A | 178 | mg/L |
| B | 53.4 | mg/L |
| C | 8.9 | mg/L |
| Reference | 0 | mg/L |

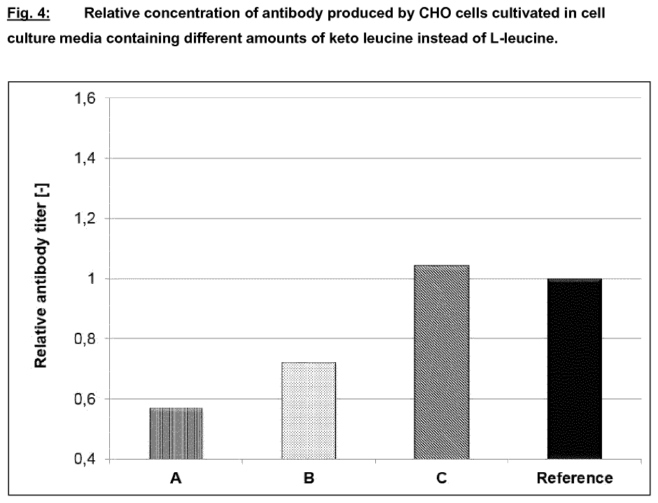

FIG. 4 shows the relative concentration of antibody produced by the CHO cells cultivated in culture media containing different amounts of keto leucine instead of L-leucine as shown in FIG. 3. The concentration of antibody produced by the cells cultivated in the reference culture medium containing L-leucine (Reference) were normalized to 1.0. This clearly shows that L-leucine can be substituted by the branched chain keto acid keto leucine in cultured CHO cells for the production of antibodies.

### Example 4

### Antibody Production in Chinese Hamster Ovary (CHO) Cells in Presence of Different Concentrations of Keto Valine

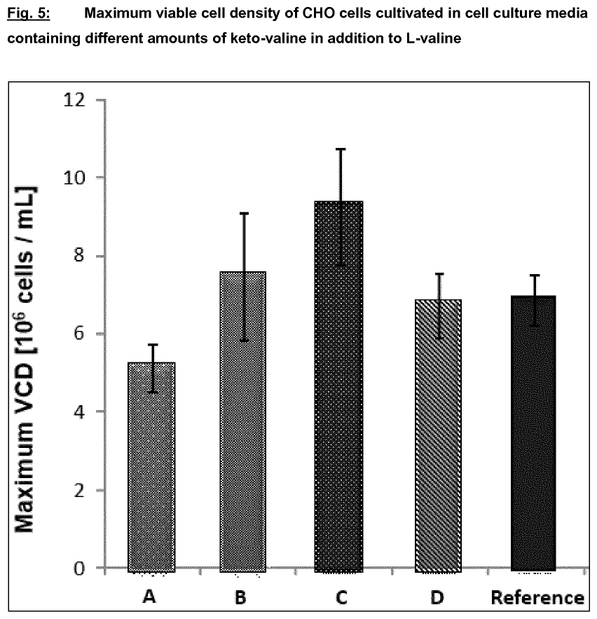

Antibody producing chinese hamster ovary (CHO) cells (subclone DG44) were cultivated in shake flasks (125 ml or 250 ml with a culture volume of 50 ml or 100 ml respectively) in TC42/CHOMACS CD culture medium (TeutoCell AG) containing 8 mM L-glutamine. The culture medium was prepared by mixing two solutions. The first contained the conventional medium formulation (containing L-valine and calcium chloride). In the second, the keto acid keto-val was added to the conventional medium at 163 mg/L as calcium salt. These solutions were mixed in different ratios. FIG. 5 shows the maximum viable cell density of CHO cells cultivated in culture media containing different amounts of keto valine in addition to L-valine. The X-axis shows the maximum viable cell density (VCD) in 10⁶ cells per ml. A represents the solution containing L-valine and keto valine at 163 mg/L. B is a mixture containing 70% (v/v) of medium with keto valine and 30% (v/v) of the conventional medium. C is a mixture of 55% (v/v) of medium with keto valine and 45% (v/v) of the conventional medium. D contains 5% (v/v) of the keto valine solution, while the reference corresponds to the conventional culture medium as described above. The resulting concentrations of keto valine in the final culture medium are shown in Table 2.

TABLE 2
Keto valine (keto-val) concentrations in the final culture medium

| | Final keto-val | |
|---|---|---|
| | concentration | |
| A | 163 | mg/L |
| B | 114.1 | mg/L |
| C | 89.65 | mg/L |
| D | 8.15 | mg/L |
| Reference | 0 | mg/L |

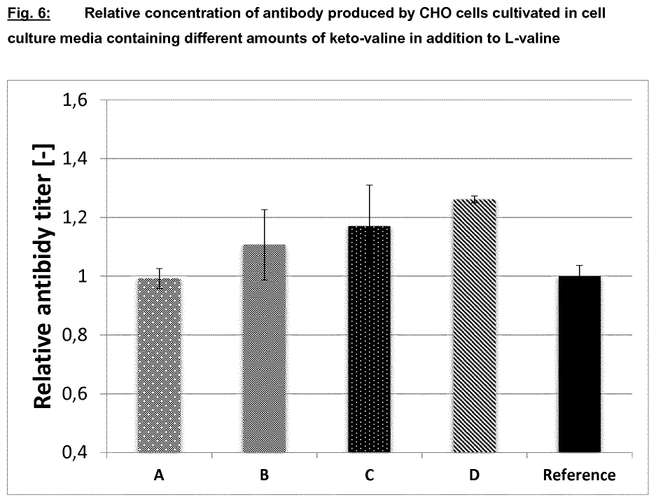

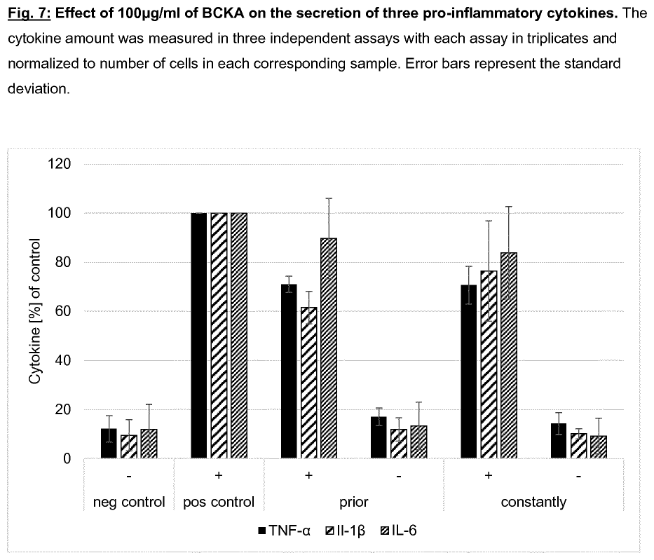

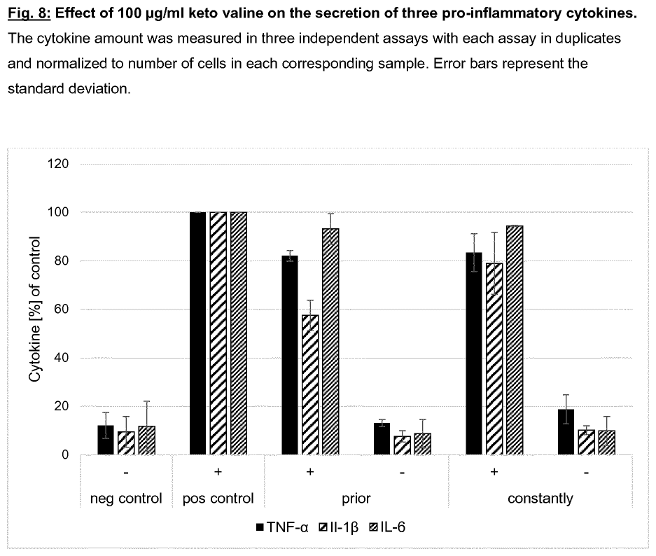

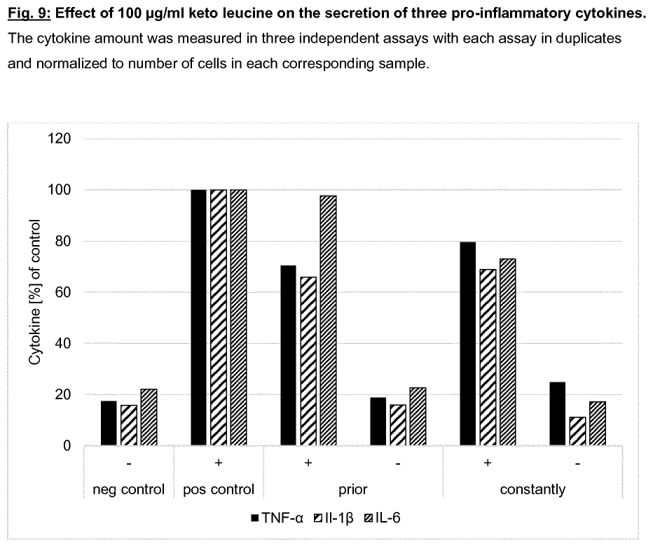

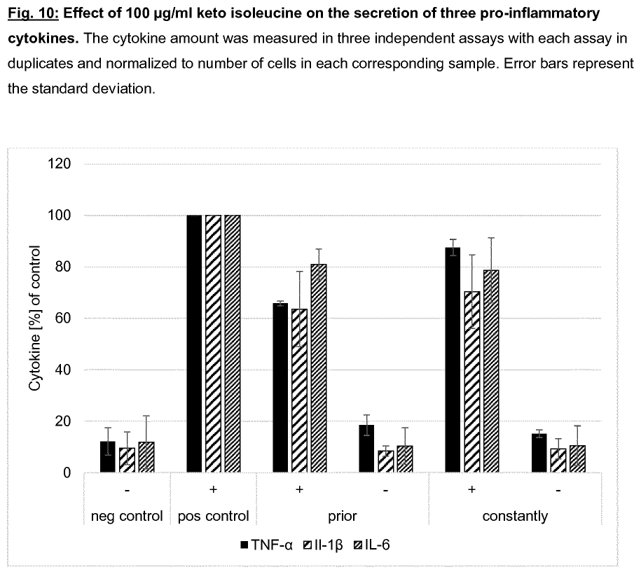

FIG. 6 shows the relative concentration of antibody produced by the CHO cells cultivated in culture media containing different amounts of keto valine and L-valine as shown in FIG. 5. The concentration of antibody produced by the cells cultivated in the reference were normalized to 1.0. It could be clearly shown that the antibody production in presence of keto valine is enhanced in comparison to the cells cultivated in absence of keto valine.

### Example 5

### Effects of BCKA on the Secretion of Cytokines

First, macrophages were obtained out of THP-1 monocytes via differentiation. When investigating the preventative scenario, a mixture of BCKA comprising Ca⁺-salts of the branched chain keto acids keto leucine : keto isoleucine : keto valine in a ratio of 2:1:1 (BCKA) was added 24 hours before inducing immune reaction through LPS. For the treatment with BCKA different time periods were chosen-prior and constantly. Treatment with BCKA stopped when LPS treatment was started (BCKA treatment prior to LPS stimulus) or treatment with BCKA was continued (BCKA treatment constantly to LPS stimulus). To evaluate the level of immune reaction, the pro-inflammatory cytokines TNF-α, IL-18 and IL-6 were measured in cell culture supernatants. The expression of pro-inflammatory cytokines of cells treated with BCKA is shown in FIG. 1.

FIG. 7 shows the effect of 100 μg/ml of BCKA on the secretion of three pro-inflammatory cytokines. The cytokine amount was measured in three independent assays with each assay in triplicates and normalized to number of cells in each corresponding sample. Error bars represent the standard deviation.

Positive and negative control were not exposed to BCKA at any time and display the cytokine release triggered by lipopolysaccharides versus the stimulated baseline. Cells that were treated with BCKA and then with LPS as an inflammatory stimulus are marked as “(+) prior to inflammation” and cells that were not exposed to LPS but likewise with the BCKA “(−) prior to inflammation”. The latter served as a control to check if BCKA itself caused cytokine release or interfered with the assay in another manner. This was excluded since cells that were treated with natural compounds showed the same cytokine profile as the negative control. In case of pre-treatment with BCKA before the LPS stimulus, a significant reduction of the cytokine release could be observed. This confirms a significant effect of the prior and constantly treatment with BCKA on the immune reaction. Furthermore, similar results were obtained using the single keto acids keto-valine, keto-leucine and keto-isoleucine.

The expression of pro-inflammatory cytokines of cells treated with keto-valine is shown in FIG. 8. FIG. 9 shows the expression of pro-inflammatory cytokines treated with keto-leucine. In FIG. 10 cells were treated with keto-isoleucine.

FIG. 8 shows the effect of 100 μg/ml keto valine on the secretion of three pro-inflammatory cytokines. The cytokine amount was measured in three independent assays with each assay in duplicates and normalized to number of cells in each corresponding sample. Error bars represent the standard deviation.

FIG. 9 shows the effect of 100 μg/ml keto leucine on the secretion of three pro-inflammatory cytokines. The cytokine amount was measured in three independent assays with each assay in duplicates and normalized to number of cells in each corresponding sample.

FIG. 10 shows the effect of 100 μg/ml keto isoleucine on the secretion of three pro-inflammatory cytokines. The cytokine amount was measured in three independent assays with each assay in duplicates and normalized to number of cells in each corresponding sample. Error bars represent the standard deviation.

1. A culture medium, comprising at least one keto acid selected from the group consisting of keto leucine, keto valine, keto isoleucine, keto phenylalanine, and a salt thereof.

2. The culture medium of claim 1, wherein the at least one keto acid is a branched chain keto acid selected from the group consisting of keto leucine, keto valine, and keto isoleucine.

3. The culture medium of claim 1, wherein the culture medium comprises keto leucine, keto valine, and keto isoleucine in an approximate ratio of 2:1:1.

4. The culture medium of claim 1, comprising an alkali metal salt or an alkaline earth metal salt of the at least one keto acid.

5. The culture medium of claim 1, comprising a Na⁺, K⁺, Ca²⁺, or Mg²⁺ salt of the at least one keto acid.

6. The culture medium of claim 1, further comprising at least one selected from the group consisting of a carbohydrate, a free amino acid, an inorganic salt, a buffering agent, and a vitamin.

7. The culture medium of claim 1, which is in form of a liquid a gel, a powder, a granulate, a pellet, a tablet.

8. The culture medium of claim 1, wherein the culture medium is a serum-free medium or a defined medium.

9. The culture medium of claim 1, wherein the at least one keto acid is present in the culture medium at a concentration in a range of from 0.01 to 10 g/L.

10. A concentrated culture medium having a 2-fold, 3-fold, 3.33-fold, 4-fold, 5-fold or 10-fold concentration of at least one keto acid selected from the group consisting of keto leucine, keto valine, keto isoleucine, keto phenylalanine, and a salt thereof, relative to the concentration of the at least one keto acid in the culture medium of claim 9.

11. A method for culturing cells, the method comprising culturing cells in the culture medium of claim 1.

12. The method of claim 11, wherein the cells are animal cells or plant cells.

13. The method of claim 12, wherein the cells are selected from the group consisting of CHO cells, COS cells, VERO cells, BHK cells, HEK cells, HELA cells, AE-1 cells, insect cells, fibroblast cells, muscle cells, nerve cells, stem cells, skin cells, endothelial cells, and hybridoma cells.

14. A method of producing a culture medium, comprising adding one or more keto acids selected from the group consisting of keto leucine, keto valine, keto isoleucine, keto phenylalanine, and a salt thereof to a culture medium.

15. A method for manufacturing a cell culture product, comprising:
providing a cell capable of producing the cell culture product;
culturing a cell capable of producing the cell culture product in the culture medium of claim 1; and
obtaining the cell culture product from the culture medium or from the cell.

16. The method of claim 15, wherein the cell culture product is at least one selected from the group consisting of a therapeutic protein, a diagnostic protein, a polysaccharide, heparin, an antibody, a monoclonal antibody, a growth factor, an interleukin, a virus, a virus-like particle, and an enzyme.
